(19) United States
(12) Patent Application Publication
(10) Pub. No.: US 2022/0323240 A1
(43) Pub. Date: Oct. 13, 2022

(54) SYSTEM CONSISTING OF SPRING ADAPTER AND ORTHOPEDIC JOINT SYSTEM, AND SPRING ADAPTER

(71) Applicant: OTTO BOCK HEALTHCARE PRODUCTS GMBH, Wien (AT)

(72) Inventors: Dirk SEIFERT, Wien (AT); Stefan HAIDER, Wien (AT)

(73) Assignee: OTTO BOCK HEALTHCARE PRODUCTS GMBH, Wien (AT)

(21) Appl. No.: 17/634,529

(22) PCT Filed: Aug. 11, 2020
(86) PCT No.: PCT/EP2020/072444

§ 371 Date: Feb. 10, 2022

(30) Foreign Application Priority Data
Aug. 13, 2019 (DE) 10 2019 121 797.0

(51) Int. Cl. A61F 2/64; A61F 5/01

(52) U.S. Cl. CPC A61F 2/64; A61F 5/0125; A61F 2002/5006

(57) ABSTRACT

A spring adapter for arrangement on an orthopedic joint system is disclosed, where the orthopedic joint system includes an upper part and a lower part articulated thereto about a pivot axis. The joint system comprises a resistance element that exerts a resistive force on a pivoting movement about the pivot axis. The spring adapter comprises at least one pneumatic spring element, flexible spring element and/or torsion spring element and is tensioned when the upper part is pivoted relative to the lower part.

The invention relates to a system consisting of a spring adapter for arrangement on an orthopedic joint device and the orthopedic joint device having an upper part and a lower part fastened thereto so as to be pivotable about a pivot axis, the joint device having a resistance device that applies a resistance to pivoting about the pivot axis. The invention also relates to a spring adapter as such, for detachable fastening to a resistance device.

Orthopedic joint devices are used in prostheses, orthoses and what are known as exoskeletons, a special case of the orthoses. Orthopedic joint devices replace missing or lost limbs in prostheses. In orthoses, the joint functions, in particular the pivotability of two limbs with respect to one another, is reproduced by the pivotability of an upper part and the lower part about a pivot axis. In the simplest case, the respective pivot axis can be designed as a one-axis, stationary joint axis; polycentric joint devices are possible and provided as an alternative thereto, and have a non-stationary position of a pivot axis. The orthopedic joint devices often are assigned resistance devices for the purposes of influencing the pivot movement, the latter being able to influence the flexion movement and/or extension movement of the upper part relative to the lower part. The resistance devices may be combined with spring elements, for example to assist an extension movement after a present flexion movement. Actuators may be assigned to these spring elements in order to facilitate a timely output of the stored energy.

Hydraulic, pneumatic and elastic spring elements are used to influence the pivot behavior of the orthopedic joint device, with the spring elements being securely installed and serving to apply restoring forces and/or store energy. The hydraulic and pneumatic, and also elastic elements are matched to one another and are designed to absorb common loads and cover the conventional load and movement spectrum.

Furthermore, there are orthopedic components that are constructed for specialist applications. Such so-called sport prostheses or sport orthoses are designed for loads under physical exertion and have spring-damper elements that assist a large scope of movement and are able to withstand high loads. By way of example, air springs or else rope-like elastomer elements are used as spring components in this case. Such systems are not suitable for daily use and as a rule are custom-made items, which are very expensive.

It is therefore an object of the present invention to provide a patient who has to resort to the use of orthopedic joint devices with an extended range of use of the orthopedic joint device, in particular in order to be able to take part in sports using the generally individually fitted orthopedic joint devices.

According to the invention, this object is achieved by a system with an orthopedic joint device with a spring adapter having the features of the main claim. Advantageous embodiments and developments of the invention are disclosed in the dependent claims, the description and the drawings.

The system consisting of a spring adapter for arrangement on an orthopedic joint device and the orthopedic joint device having an upper part and a lower part fastened thereto so as to be pivotable about a pivot axis, with the joint device having a resistance device that applies a resistance to pivoting about the pivot axis, provides for the spring adapter to comprise at least one compression spring element, bending spring element, tension spring element or torsion spring element, which is tensioned, that is to say compressed, bent, pulled or twisted depending on the design of the spring element, when the upper part is pivoted relative to the lower part. By means of the spring adapter, which is designed as a parallel-elastic element in relation to the remaining components of the joint device, it is possible to extend the functionality of the conventional prostheses or orthoses, in particular for sporting activities such as skiing or the like, such that the additional arrangement of the spring adapter on the orthopedic joint device can increase the range of use of the orthopedic device for the respective user in cost-effective fashion. At the same time, the orthopedic joint device remains unchanged for daily care, and so it is possible to realize a cost-effective use situation that is advantageous for the patient.

The spring adapter may comprise a distal abutment portion and a proximal abutment portion which in the assembled state abut or at least in the case of compression abut against distal and proximal abutment regions of the joint device and/or the resistance device. Preferably, the abutment portions abut against the abutment regions during the whole use of the joint device and the spring adapter and the whole scope of use of the joint device, in order to avoid play, jerky engagement or bothersome rattling noises.

The spring adapter may comprise at least one tube spring, helical spring, air spring, Belleville washer, Belleville washer assembly and/or an elastomer element in order to provide the additional spring force required in each case. As a result, it is possible to provide sufficient resistance to an extraordinary and elevated load which is in the range of non-daily use.

The abutment portion, by means of which the spring adapter can be secured between two movable components, can be formed as part of the spring element. Alternatively, the respective abutment portion can be fastened to the spring element. The spring element, for example the compression spring element or the spring adapter, is arranged between two movable components of the orthopedic joint device, for example between the upper part and the lower part or in the region of the resistance device between a housing of the resistance device and the upper part or a receptacle, for example on a piston rod, if the resistance device is designed as a hydraulic linear damper. The spring element or the entire spring adapter can be assembled and removed by the respective user of the system. Elastomer elements in particular may consist of different materials and may be put together to form stacks of elastomer elements in order to produce different characteristics. By way of example, it is possible to form elastomer elements as annular disks which are slotted in the radial direction, are stacked one above the other and, for example, can be arranged around the piston rod or another guide. Different elasticity properties and stiffnesses of different elastomer disks facilitate a simple adjustment and variation of the characteristic of the compression spring element or of the spring adapter by way of a suitable composition of the respective disk assemblies.

The abutment portion may comprise a holding device for securing it to the abutment region or the joint device in interlocking fashion, for example to prevent twisting, displacing or tilting of the compression spring element or of the entire spring adapter relative to the remaining components of the joint device. By way of example, the holding device may be in the form of an undercut, a cone, a peg or a projection, which is formed in a corresponding projection or in a corresponding recess on or in the holding region.

Advantageously, the spring adapter is secured to the joint device and/or the resistance device and in interchangeable, and hence removable and re-applicable, fashion. As a result, it is possible to match the spring characteristic or the spring stiffness to the respective requirements or needs by using the suitable spring adapter.

The resistance device may comprise a housing, for example a damper housing, in which a piston divides a fluid chamber or a cylinder into a flexion chamber and an extension chamber. If the resistance device is in the form of a hydraulic linear damper, it has a piston rod that protrudes out of the damper housing. The spring adapter comprises a fastening device for securing it to the joint device and/or the resistance device, for example to the piston rod. If the spring adapter is arranged at the piston rod, the spring adapter and the compression spring or a plurality of compression spring elements are firstly assigned to the components movable relative to one another and are secondly guided and affixed such that stabilization of the entire spring adapter is achieved.

A plurality of compression springs may be arranged around the piston rod, for example be arranged around the latter in symmetric fashion or else with different spacings therebetween or be applied next to the piston rod with an axial offset. It is likewise possible for the compression spring element or for a plurality of compression spring elements to be arranged coaxially around the piston rod such that the piston rod is surrounded over at least some of its perimeter by the spring element, the spring elements or the spring adapter. The piston rod is then at least partly received by the compression spring elements surrounding it.

The fastening device for securing the spring adapter to the piston rod is preferably designed for reversibly securing the spring adapter to the piston rod in interlocking fashion and may for example have resilient components that allow the spring adapter or the compression spring elements to be secured while applying an elastic peripheral force at the piston rod or between the upper part and the damper housing. Hinges or clip-on devices may be arranged or formed to interchange the spring adapter or individual spring elements and fasten these to the piston rod.

A development of the invention provides for a plurality of individual springs to be arranged in the compression spring element and connected in parallel. The individual springs themselves may consist of series-connected individual spring components, for example elastomer disks or the like. The individual springs connected in parallel may have different spring characteristics and/or different engagement points for forming a gradated spring behavior. Thus, it is possible that initially a comparatively soft spring engages from the outset and a stiffer spring or a plurality of stiffer springs is or are compressed after a predetermined displacement travel has been reached in order thus to be able to provide a progressive overall spring characteristic.

As an alternative to or complementing the individual springs connected in parallel, the spring adapter may comprise at least one spring element with different spring stiffnesses connected in series in order to provide a resistance that varies over the deformation travel.

The resistance device may comprise a hydraulic and/or pneumatic damper, the orthopedic joint device being designed in particular as an orthosis joint or prosthesis joint, more particularly as an artificial knee joint.

The spring adapter may have an adjustable form, for example by virtue of displacing the spring characteristic by changing the pretension or by displacing the zero point. Likewise, the stiffness may be designed to be variable, for example by replacing spring elements or by virtue of varying the spring length and/or stiffness by way of an adjustment element or spacer.

The invention likewise relates to a spring adapter for detachable fastening to a resistance device, as explained above. Encapsulating a plurality of compression spring components or compression spring elements in a housing may be advantageous. Different forms may be realized when embodying the spring adapter or the compression spring elements or torsion spring elements as elastomer elements, as a result of which the frequently limited installation volume can be optimally utilized. By way of example, the compression spring elements or the spring adapter may have a U-shaped design, the open sections of the U-shaped compression spring elements optionally being arranged so as to be twisted in relation to one another in the case of elastomer components stacked one above the other, in order to obtain a uniform load and prevent a tilting moment. The characteristics of the spring elements are selectable according to the application; they may be linear, and degressive or progressive.

An identification device which is coupled to the resistance device can be arranged on the spring adapter and/or the joint device. By way of example, the identification device may be an RFID chip or a sensor or a contact, which is closed after the spring adapter is attached. There can be a modified damping behavior of the resistance device as soon as the spring adapter being arranged on the orthopedic joint device is recognized automatically. By way of example, in the case of an electronically controlled, adjustable resistance device, an appropriate program may be saved in the control unit, the program bringing about a stronger or angle-dependently adapted damping or another type of adjustment of the resistances. The identification device facilitates an automatic adjustment of the damper behavior to the additional spring elements, or else a return to the basic setting after the spring adapter has been removed.

In addition to an elastic behavior of the spring adapter or the spring elements, it is also possible to set a speed-proportional behavior by way of the viscous properties of viscous elements or of a hydraulic fluid.

Attachment to structures already present in the joint device can be achieved by removable spindles, by an interlocking fit or by clamping on components that are already present.

Exemplary embodiments of the invention are explained in more detail below on the basis of the attached drawings, in which:

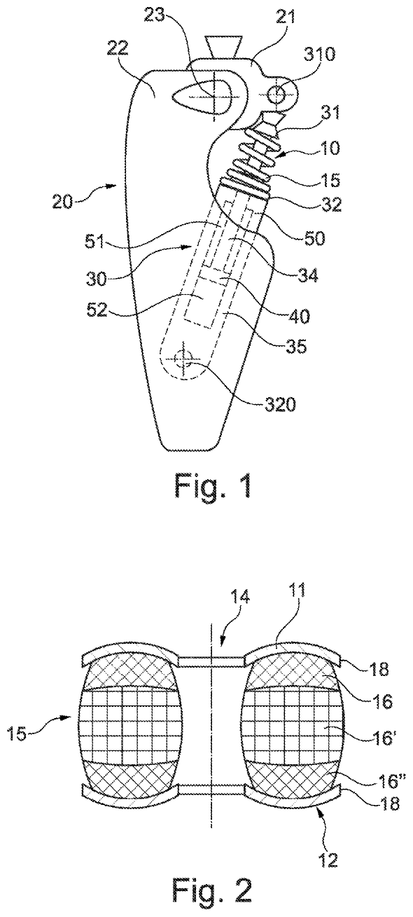

FIG. 1 shows a schematic illustration of a system consisting of a spring adapter and joint device;

FIG. 2 shows a schematic illustration of a multi-layer elastomer element;

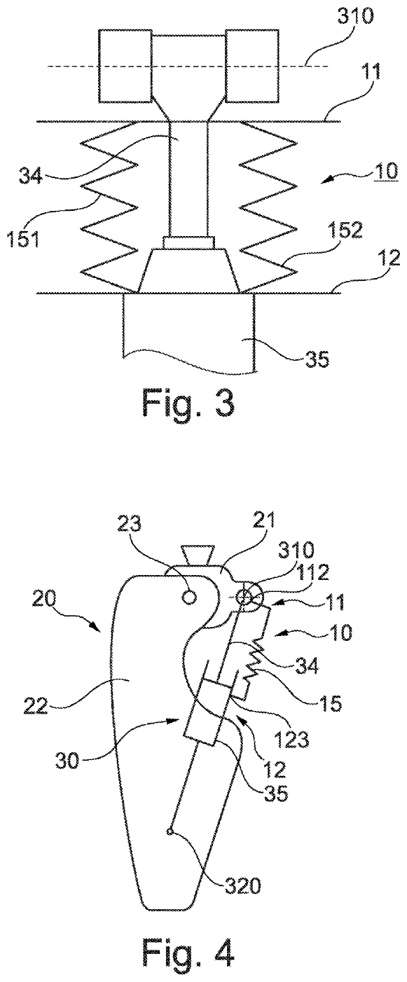

FIG. 3 shows a schematic illustration of a spring adapter with two axially offset spring elements;

FIG. 4 shows a schematic illustration of a variant of the arrangement of the spring element;

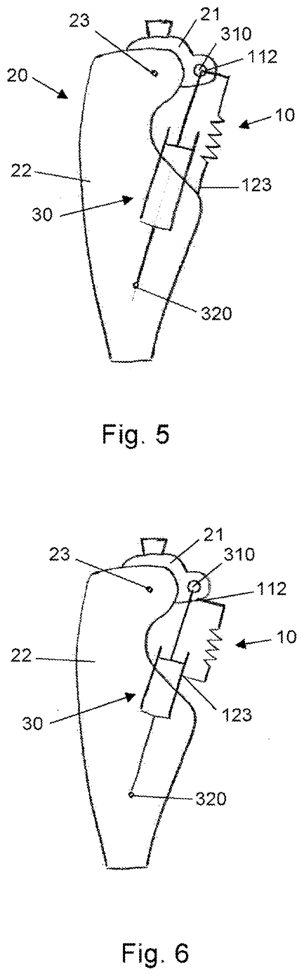

FIG. 5 shows a variant of FIG. 4 with a connection to the lower part;

FIG. 6 shows a further variant of FIG. 4 with a connection to a resistance housing and an upper part;

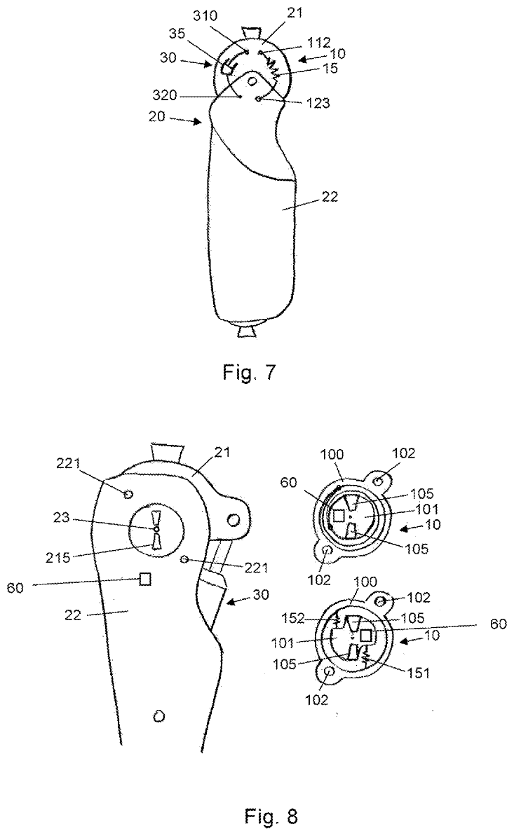

FIG. 7 shows a variant with a rotation hydraulics unit;

FIG. 8 shows a variant with a coaxial connection of the spring adapter; and

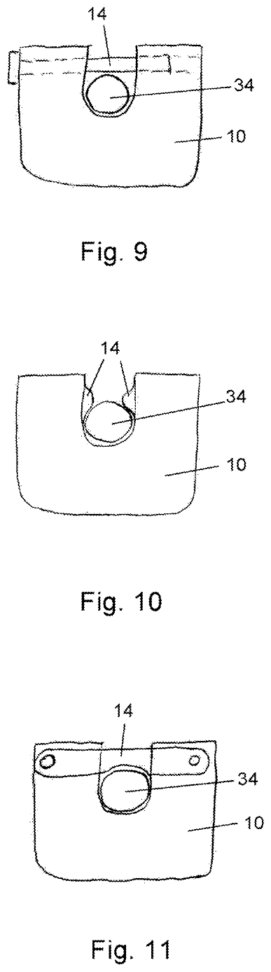

FIGS. 9-11 show schematic illustrations of options for fastening the spring adapter around the piston rod.

FIG. 1 shows an orthopedic joint device 20 in the form of a prosthetic knee joint in a schematic side view. The orthopedic joint device 20 comprises an upper part 21 and a lower part 22 fastened thereto, the two parts being mounted on one another so as to be pivotable about a pivot axis 23. A resistance device 30 is arranged between the upper part 21 and the lower part 22 and influences the pivoting movement about the pivot axis 23. The resistance device 30 may be in the form of a purely passive resistance device, for example a fluid damper, more particularly a hydraulic damper. Moreover, the resistance device 30 may be provided with a magnetorheological fluid. An actuator in an appropriate circuit may likewise provide the appropriate resistance. Thus, a drive in generator operation may be used as a resistance device 30. In the exemplary embodiment illustrated, the resistance device 30 is mounted so as to be pivotable about two bearing spindles 310, 320. The proximal connection point of the resistance device 30 on the proximal bearing spindle 310 is situated on the back side of the upper part 21 in the direction of walking; the distal bearing spindle 320 is arranged or formed on the lower part 22.

In FIG. 1, the orthopedic joint device 20 is shown in a maximally extended position. Further pivoting of the upper part 21 in the extension direction, counterclockwise in the illustrated configuration, is no longer possible. Should flexion be performed, the upper part 21 is pivoted clockwise about the pivot axis 23 or, in the case of a stationary upper part 21, the lower part 22 is pivoted counterclockwise about the pivot axis 23. An appropriate force is applied to the resistance device 30 in the form of a hydraulic damper with linear action so that the upper bearing spindle 310 is moved in the direction of the lower bearing spindle 320. The resistance device 30 comprises a housing 35, in which a piston 40 is mounted on a piston rod 34 that protrudes from said housing 35. The piston 40 divides a cylinder 50 formed within the housing 35 into a flexion chamber 51 and an extension chamber 52, which are flow-connected to one another. The flow resistance between the flexion chamber 51 and the extension chamber 52 can be set and, in particular, adjusted on the basis of movement states or sensor data by means of one or more valves that are not illustrated here. To this end, a control device is coupled to a computer or a processor with an adjustment device for the valves such that the valves can be opened or closed depending on movement data, for example pivoting speeds, accelerations, torques, forces or the like.

Abutment regions 31, 32 are arranged or formed on the resistance device 30 as abutment faces, with a spring adapter 10 being arranged therebetween. A distal abutment face 32 is arranged or formed in the region of the housing 35, on a damper housing of a hydraulic damper in the present case, and a proximal abutment face 31 is fastened to or formed on the proximal end of the piston rod 34. The two abutment faces 31, 32 serve to absorb forces that occur during a compression or relaxation of the spring adapter 10. In the exemplary embodiment illustrated, the spring adapter 10 comprises a compression spring 15 in the form of a helical spring or spiral coil spring, but other compression spring types may likewise be provided, for example elastomer elements or the like. In this case, it is not necessary for the spring adapter to also abut against the two abutment surfaces 31, 32 in the maximally extended state and exert a pressure force in the extension direction. There also is the option, for example for skiing, of the spring adapter 10 only being compressed after a certain angle position has been reached in the case of a flexed joint. By way of example, the spring element 15 arranged in the spring adapter 10 may only be in a neutral position at a flexion angle of 15° and only be compressed in the case of further flexion. A tensile load would be exerted between the upper part 21 and the lower part 22, up to the adjustable angle of the neutral position, by way of the resistance device 30 and the spring adapter 10. Then, the spring adapter 10 would be arranged between the abutment regions 31, 32 in tensile force-transmitting and compression force-transmitting fashion.

Instead of having a single compression spring 15, it is possible that a plurality spring elements form the spring adapter 10 or are part of the spring adapter 10. Different spring types may be combined in mixed fashion in a spring adapter 10, for example a plurality of helical springs or a helical spring in combination with one or more Belleville washers or an elastomer element. The spring adapter 10 is designed to be detachably securable to the resistance device 30 in easily replaceable fashion, preferably in a way that avoids having to separate the resistance device 30 from the joint device 20.

FIG. 2 shows a variant of the spring adapter 10 in a detailed view. At its upper, proximal end the spring adapter 10 has a proximal abutment portion 11 and at its lower, distal end it has a distal abutment portion 12, which portions are each embodied as part of a Belleville washer 18 or a Belleville washer element 18 in the exemplary embodiment illustrated. A combination of a plurality of elastomer elements 16, 16′, 16″ is arranged between the two Belleville washer elements 18 or Belleville washers 18 as compression spring element 15. The elastomer elements 16, 16′, 16″ may be stacked on one another or be connected to one another, for example by adhesive bonding. The elastomer elements or spring elements have a cutout in their center, into which the piston rod 34 of a hydraulic damper, for example, can be inserted. The cutout is formed such that the piston rod 34 is at least partly surrounded by the spring adapter 10 in the assembled state. It may likewise be possible, for example by way of a hinged solution, to fold open the spring adapter 10 and place it around the piston rod. A fastening device 14, by means of which the spring adapter 10 can be secured in reversible and interlocking fashion to the piston rod 34, may be arranged or embodied on the spring adapter 10. The fastening device 14 may be in the form of a bar, bolt, spring element or else magnet, by means of which two portions or regions of the spring adapter 10 that are arranged so as to be able to move relative to one another are held against one another in order to avoid an unwanted detachment from the piston rod 34 or the resistance device 30 overall. The abutment portions 11, 12 are preferably formed such that they are formed in corresponding fashion to the abutment regions 31, 32; they may also have an interlocking engagement with the abutment regions 31, 32 in order to prevent a relative movement, for example twisting about the longitudinal extent of the resistance device 30. By way of example, the abutment portions 11, 12 may snap into or catch on the abutment regions 31, 32.

FIG. 3 shows a variant of the spring adapter 10 which has two abutment portions 11, 12, the proximal abutment portion 11 of which abuts against a thickening of the piston rod 34 and, in the case of flexion of the joint (not illustrated here) and loading of the piston rod 34 with a pressure force, tensions and compresses the two spring elements 151, 152. The counter bearing in this respect is the distal abutment portion 12, which is supported by the housing 35 of the resistance device 30. Further spring elements may be arranged between the two abutment portions 11, 12 and these further spring elements may be arranged so as to have an effect in parallel with the spring elements already present. As an alternative to or complementing this, a plurality of different spring elements may be arranged in succession so as to act in series. It is likewise possible that additional spring elements become active and are compressed only after a certain displacement travel of the two abutment portions 11, 12 toward one another, for example by virtue of an elastomer element or tube damper initially being arranged on the distal abutment portion 12 at a distance from the proximal abutment portion 11.

FIG. 4 illustrates a variant of the arrangement of the spring adapter 10 on the joint device 20. The proximal abutment portion 11 is arranged on the proximal bearing spindle 310 of the piston rod 34 on the upper part 21 in the exemplary embodiment illustrated. The distal abutment portion 12 is situated on the housing 35 of the resistance device 30, which may be embodied as a hydraulic damper or pneumatic damper, for example. Respective holding devices 112, 123 are arranged or formed on both abutment portions 11, 12, the spring adapter 10 being able to be interlockingly securable to the joint device 20, for example the resistance device 30, by means of said holding devices. The holding devices 112, 123 may be through-bores for screws or bolts and may be formed with or without a thread. Likewise, the holding devices 112, 123 may be designed to be inserted into and latch on corresponding cutouts on the upper part 21 and the housing 35, for example the lower part 22.

A further variant is illustrated in FIG. 5, the proximal connection being embodied like in FIG. 4 in this case. The distal connection is implemented at the lower part 22 by way of the distal holding device 123 on the distal abutment portion 12. In the embodiment according to FIG. 6, the distal connection is implemented as described in FIG. 4, but the proximal connection is not implemented in the region of the bearing spindle 310 but at the lower side of the upper part 21 by way of the proximal holding device 112.

FIG. 7 shows a further variant in which a rotation hydraulics unit or rotationally effective resistance device 30 is provided instead of a linearly effective resistance device 30, for example a linearly acting hydraulic damper. Instead of hydraulic damping by way of valves or chokes in a flow channel, the resistance device 30 can also be realized by way of magnetorheological fluids or drives in generator operation. The spring adapter 10 is arranged on and secured to the upper part 21 and the lower part 22. In the exemplary embodiment illustrated, the holding devices 112, 123 are pegs or screws, for example, which are arranged on identical circular trajectories about the pivot axis 23 such that the two holding devices 112, 123 are moved toward one another when the upper part 21 is pivoted relative to the lower part 22. However, in principle it is also possible that the holding devices 112, 123 are each arranged at different radii in relation to the pivot axis 23 so that shearing forces, bending forces and/or, in the case of an alternative embodiment of the spring element, torsion forces also occur in addition to a pure compression force of the spring element 15, said additional forces being absorbed and stored by an appropriate embodiment of the spring element 10. The spring adapter 10 may also be designed as a spring adapter that is effective in relation to torsion or bending and may have a torsion spring or a bending spring or a plurality of springs. It is likewise possible for a plurality of different spring types, for example torsion springs and bending springs, to be combined with one another.

A further variant is shown in FIG. 8, within the scope of which the left drawing illustrates the joint device 20 with a resistance device 30 and the right drawings illustrate two embodiments of the spring adapter 10. The basic structure of the joint device 20 corresponds to that of FIG. 1 and FIGS. 4 to 6. Instead of an arrangement of the spring adapter 10 between two abutment portions that approach one another, FIG. 8 provides for a coaxial connection of the spring adapter 10. Two pegs or bores 221 are provided in/on the joint device 20 and these are correspondingly positioned in relation to bores or pegs 102 on/in a first housing part 100 of the spring adapter 10. By way of the pegs or bores 221, which may be provided with threads, it is possible to interlockingly secure the first housing part 100 to the joint device 20, for example by plugging on and locking and/or by screwing. In the exemplary embodiment illustrated, securing is implemented at the lower part 22. Interlock devices 215 are arranged or formed in the upper part 21 on both sides of the pivot axis 23. The interlock devices may also be arranged or formed on the upper part 21 at different distances from the pivot axis 23, and it is likewise possible for only one interlock device 215 or more than two interlock devices 215 to be provided. The interlock devices 215 may be embodied as projections, depressions, toothings or bores, into which in the assembled state correspondingly shaped interlock elements 105 engage on a second housing part 101 that is mounted so as to be rotatable relative to the first housing part 100. In the variant of the spring adapter 10 illustrated at the top, a spiral spring or torsion spring 105 is situated between the two housing parts 100, 101, while two or more compression spring elements 151, 152 are arranged in the lower exemplary embodiment of the spring adapter 10. If the upper part 21 is pivoted relative to the lower part 22 about the pivot axis 23 in the assembled state, are twisted relative to the lower part 22 and the first housing part 100 by way of the interlock elements 105, 215 since the first housing part 100 is secured in stationary and non-rotatable fashion on the lower part by way of the interlocking connection, for example by way of pegs and bores 102, 221. The second housing part 101 twists relative to the first housing part 100 and the spring elements 151, 152 or the spring element 15 is or are tensioned.

In the exemplary embodiment according to FIG. 8, identification devices 60 which automatically detect the presence or lack of spring adapter 10 are arranged both on the spring adapter 10 and on the joint device 20. If a spring adapter 10 is present and assembled, an appropriate signal is transmitted from the identification device 60 to a control device (not illustrated) so that it is possible to implement different damping settings or resistance settings in the resistance device 30. The settings may differ depending on the utilized spring adapter 10. By way of example, if very strong spring elements are used in order to absorb particularly high loads, for example during skiing, the resistance device 30 may provide appropriately modified resistances. In an embodiment as a hydraulic damper, the degree of damping and the damping progression may be adjusted depending on the spring resistance by the spring adapter 10. Detection can be implemented by way of an RFID element, a magnetic code or any other sensor element. As a result, it is possible for the orthopedic joint device 20 to automatically set and adjust the damping program or resistance program on the basis of the chosen spring adapter 10.

FIGS. 9 to 11 illustrate schematic sectional illustrations regarding different options for fixing the spring adapter 10 around a piston rod 34. In FIG. 9, the spring adapter 10 or the spring assembly is secured by way of a bolt as a fastening device 14. A U-shaped cutout for receiving the piston rod 34 is provided in the spring assembly of the spring adapter 10. Moreover, a thread and a through-hole are arranged in the spring adapter 10 such that, following the assembly of the spring adapter 10 around the piston rod 34, the insertion opening of the cutout is sealed by inserting and screwing in the bolt 14, and the spring adapter is securely fixed to the piston rod 34.

In FIG. 10, the fastening device 14 is in the form of an elastic locking mechanism with elastic projections made of an elastomer or leaf springs or the like. As a result, the spring adapter 10 can be installed and removed without tools.

In FIG. 11, the fastening device 14 is in the form of a folding lever, which is pivotably mounted on the spring adapter 10 and which can be sealed after the insertion of the piston rod 34.

1. A system comprising a spring adapter and an orthopedic joint device, the spring adapter being configured to be arranged on an orthopedic joint device wherein the orthopedic joint device has an upper part and a lower part fastened thereto so as to be pivotable about a pivot axis, the joint device having a resistance device that applies a resistance to pivoting about the pivot axis, wherein the spring adapter comprises at least one compression spring element, bending spring element and/or torsion spring element and is tensioned when the upper part is pivoted relative to the lower part.

2. The system as claimed in claim 1, wherein the spring adapter comprises a distal abutment portion and a proximal abutment portion which in the assembled state abut against distal and proximal abutment regions of the joint device and/or the resistance device in the case of a compression.

3. The system as claimed in claim 1, wherein the spring adapter comprises at least one tube spring, helical spring, air spring, Belleville washer and/or an elastomer element.

4. The system as claimed in claim 2, wherein the proximal abutment portion and the distal abutment portion is are formed as part of the spring element or fastened thereto.

5. The system as claimed in claim 2, wherein the proximal abutment portion and the distal abutment portion comprise a holding device for securing to the joint device in an interlocking fashion.

6. The system as claimed in claim 1, wherein the spring adapter is secured in interchangeable/removable fashion to the joint device and/or the resistance device.

7. The system as claimed claim 1, wherein the resistance device comprises a housing in which a piston divides a fluid chamber into a flexion chamber and an extension chamber, and wherein the spring adapter comprises a fastening device for securing it to the joint device.

8. The system as claimed in claim 7, wherein the resistance device is embodied as a damper comprising a piston rod which protrudes from the housing and on which a piston divides a fluid chamber in the housing into a flexion chamber and an extension chamber, and the fastening device is designed to secure the spring adapter to the piston rod.

9. The system as claimed in claim 8, wherein a plurality of compression springs are arranged around the piston rod or wherein the compression spring element in the assembled state surrounds the piston rod over at least some of its perimeter.

10. The system as claimed in claim 7, wherein the fastening device is designed for reversibly securing the spring adapter to the piston rod in interlocking fashion or between the upper part and the housing.

11. The system as claimed in claim 1, wherein a plurality of individual springs are arranged in the spring adapter and connected in parallel.

12. The system as claimed in claim 11, wherein the individual springs connected in parallel have different spring characteristics and/or different engagement points for forming a gradated spring behavior.

13. The system as claimed in claim 1, wherein the spring adapter comprises at least one spring element with different spring stiffnesses connected in series.

14. The system as claimed in claim 1, wherein the resistance device comprises a hydraulic and/or pneumatic damper and the orthopedic joint device is designed as an orthosis joint or prosthesis joint.

15. The system as claimed in claim 1, wherein the spring adapter has an adjustable form.

16. The system as claimed in claim 1. wherein the spring adapter comprises a first housing part that is capable of being coupled to the upper part and a second housing part that is capable of being coupled to the lower part or the resistance device, at least one spring element being arranged between these housing parts so as to be able to be tensioned.

17. The system as claimed in claim 16, wherein interlocking devices are arranged or formed on the housing parts for the purposes of securing these to the upper part and the lower part or the resistance device.

18. The system as claimed in claim 1, wherein an identification device which is coupled to the resistance device is arranged on the spring adapter and/or the joint device.

19. The spring adapter as claimed in claim 1 for detachable fastening to a resistance device or joint device.

20. A an orthopedic joint device comprising:
an upper part and a lower part fastened thereto so as to be pivotable about a pivot axis;
a resistance device that applies a resistance to pivoting about the pivot axis; and
a spring adapter comprising at least one compression spring element, bending spring element and/or torsion spring element; and further comprising a distal abutment portion and a proximal abutment portion which abut distal and proximal abutment regions of the joint device and/or the resistance device in an assembled state under compression;
wherein the proximal abutment portion and the distal abutment portion are formed as part of the spring element or fastened thereto; and wherein the spring adapter is tensioned when the upper part is pivoted relative to the lower part.
